(19) United States
(12) Patent Application Publication
(10) Pub. No.: US 2010/0173879 A1
(43) Pub. Date: Jul. 8, 2010

(54) USE OF A COMPOUND ANTAGONIST TO THE NK2 RECEPTORS OF NEUROKININ A FOR THE PREPARATION OF DRUGS USEFUL FOR PREVENTING AND TREATING OF SEXUAL DYSFUNCTION

(75) Inventors: Lisa ARVANITIS, Bridgewater, NJ (US); Christiane L'HERITIER, Paris (FR)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: SANOFI-AVENTIS, Paris (FR)

(21) Appl. No.: 12/537,345

(22) Filed: Aug. 7, 2009

Related U.S. Application Data
(63) Continuation of application No. PCT/FR2008/000135, filed on Feb. 4, 2008.

(30) Foreign Application Priority Data
Feb. 7, 2007 (FR) 0700856

(51) Int. Cl. A61K 31/568; A61K 31/5377; A61K 31/445; A61K 31/496; A61K 31/4985; A61K 31/435; A61P 15/00

(52) U.S. Cl. 514/171; 514/235.5; 514/331; 514/252.16; 514/250; 514/284

(57) ABSTRACT

The invention relates to the use of a compound antagonist to the NK₂ receptors of A neurokinine for the preparation of drugs useful for preventing and treating sexual dysfunction.

The present invention relates to the use of a compound that is a neurokinin A NK2 receptor antagonist for the preparation of medicaments for use in the prevention and treatment of sexual dysfunctions.

According to the present invention, the term “neurokinin A NK2 receptor antagonist” is intended to mean a compound chosen from:
(S)-(−)-N-[4-(4-acetamido-4-phenylpiperidin-1-yl)-2-(3,4-dichlorophenyl)butyl]-N-methylbenzamide, the international nonproprietory name (INN) of which is saredutant, and its pharmaceutically acceptable salts described in patent EP 0 474 561 and U.S. Pat. No. 5,236,921, and also in patent EP 1 173 179 for its activity in major depressive disorders;
(+)-N-[1-[2-[2-(3,4-dichlorophenyl)-5-oxo-4-phenylmorpholin-2-yl]ethyl]-4-(3-fluorophenyl)piperidin-4-yl]acetamide and its pharmaceutically acceptable salts described in the international application WO 2006/021 654; and
(+)-1′-[2-[4-benzoyl-2-(3,4-dichlorophenyl)morpholin-2-yl]ethyl]-N,N-dimethyl-1,4′-bipiperidine-4′-carboxamide and its pharmaceutically acceptable salts, which also have the property of being a neurokinin B NK3 receptor antagonist. This compound and its salts are described in international application WO 02/094 821.

It has now been found that a neurokinin A NK2 receptor antagonist chosen from saredutant and its pharmaceutically acceptable salts, (+)-N-[1-[2-[2-(3,4-dichlorophenyl)-5-oxo-4-phenylmorpholin-2-yl]ethyl]-4-(3-fluorophenyl)piperidin-4-yl]acetamide and its pharmaceutically acceptable salts and (+)-1′-[2-[4-benzoyl-2-(3,4-dichlorophenyl)morpholin-2-yl]ethyl]-N,N-dimethyl-1,4′-bipiperidine-4′-carboxamide and its pharmaceutically acceptable salts are of use in the prevention and treatment of sexual dysfunctions.

The term “sexual dysfunctions” is intended to mean any pathologies as defined by the American Psychiatric Association—DSM-IV-TR, Diagnostic and Statistical Manual of Mental Disorders, 4th edition, revised text (Washington D.C., 2000), pages 617-654 and which include disorders of sexual desire (i.e. the disorder: reduced sexual desire, and the disorder: sexual aversion), disorders of sexual arousal (i.e. female sexual arousal disorder and male erectile disorder), orgasmic disorders (i.e. female orgasmic disorder, male orgasmic disorder and premature ejaculation), painful sexual disorders (i.e. dyspareunia and vaginismus), sexual dysfunction due to a general medical condition, substance-induced sexual dysfunction, and unspecified sexual dysfunctions.

Thus, according to one of its aspects, a subject of the present invention is the use of a compound that is a neurokinin A NK2 receptor antagonist, chosen from saredutant and its pharmaceutically acceptable salts, (+)-N-[1-[2-[2-(3,4-dichlorophenyl)-5-oxo-4-phenylmorpholin-2-yl]ethyl]-4-(3-fluorophenyl)piperidin-4-yl]acetamide and its pharmaceutically acceptable salts and (+)-1′-[2-[4-benzoyl-2-(3,4-dichlorophenyl)morpholin-2-yl]ethyl]-N,N-dimethyl-1,4′-bipiperidine-4′-carboxamide and its pharmaceutically acceptable salts, for the preparation of medicaments of use in the prevention and treatment of sexual dysfunctions. In particular, a subject of the present invention is the use of saredutant and of its pharmaceutically acceptable salts, for the preparation of medicaments of use in the prevention and treatment of sexual dysfunctions.

Also in particular, a subject of the present invention is the use of (+)-N-[1-[2-[2-(3,4-dichlorophenyl)-5-oxo-4-phenylmorpholin-2-yl]ethyl]-4-(3-fluorophenyl)piperidin-4-yl]acetamide and its pharmaceutically acceptable salts, for the preparation of medicaments of use in the prevention and treatment of sexual dysfunctions.

Also in particular, a subject of the present invention is the use of (+)-1′-[2-[4-benzoyl-2-(3,4-dichlorophenyl)morpholin-2-yl]ethyl]-N,N-dimethyl-1,4′-bipiperidine-4′-carboxamide and its pharmaceutically acceptable salts, for the preparation of medicaments of use in the prevention and treatment of sexual dysfunctions.

Also in particular, a subject of the present invention is the use of saredutant and its pharmaceutically acceptable salts, of (+)-N-[1-[2-[2-(3,4-dichlorophenyl)-5-oxo-4-phenylmorpholin-2-yl]ethyl]-4-(3-fluorophenyl)piperidin-4-yl]acetamide and its pharmaceutically acceptable salts and of (+)-1′-[2-[4-benzoyl-2-(3,4-dichlorophenyl)-morpholin-2-yl]ethyl]-N,N-dimethyl-1,4′-bipiperidine-4′-carboxamide and its pharmaceutically acceptable salts, for the preparation of medicaments of use in the prevention and treatment of sexual desire disorder, more particularly of the disorder: reduced sexual desire or of the disorder: sexual aversion.

Also in particular, a subject of the present invention is the use of saredutant and of its pharmaceutically acceptable salts, of (+)-N-[1-[2-[2-(3,4-dichlorophenyl)-5-oxo-4-phenylmorpholin-2-yl]ethyl]-4-(3-fluorophenyl)piperidin-4-yl]acetamide and its pharmaceutically acceptable salts and of (+)-1′-[2-[4-benzoyl-2-(3,4-dichlorophenyl)-morpholin-2-yl]ethyl]-N,N-dimethyl-1,4′-bipiperidine-4′-carboxamide and its pharmaceutically acceptable salts, for the preparation of medicaments of use in the prevention and treatment of disorders of sexual arousal, more particularly female sexual arousal disorder and male erectile disorder.

Also in particular, a subject of the present invention is the use of saredutant and of its pharmaceutically acceptable salts, of (+)-N-[1-[2-[2-(3,4-dichlorophenyl)-5-oxo-4-phenylmorpholin-2-yl]ethyl]-4-(3-fluorophenyl)piperidin-4-yl]acetamide and its pharmaceutically acceptable salts and of (+)-1′-[2-[4-benzoyl-2-(3,4-dichlorophenyl)-morpholin-2-yl]ethyl]-N,N-dimethyl-1,4′-bipiperidine-4′-carboxamide and its pharmaceutically acceptable salts, for the preparation of medicaments of use in the prevention and treatment of orgasmic disorders, more particularly female orgasmic disorder, male orgasmic disorder and premature ejaculation.

Also in particular, a subject of the present invention is the use of saredutant and of its pharmaceutically acceptable salts, of (+)-N-[1-[2-[2-(3,4-dichlorophenyl)-5-oxo-4-phenylmorpholin-2-yl]ethyl]-4-(3-fluorophenyl)piperidin-4-yl]acetamide and its pharmaceutically acceptable salts and of (+)-1′-[2-[4-benzoyl-2-(3,4-dichlorophenyl)-morpholin-2-yl]ethyl]-N,N-dimethyl-1,4′-bipiperidine-4′-carboxamide and its pharmaceutically acceptable salts, for the preparation of medicaments of use in the prevention and treatment of painful sexual disorders, more particularly dyspareunia and vaginismus.

Also in particular, a subject of the present invention is the use of saredutant and of its pharmaceutically acceptable salts, of (+)-N-[1-[2-[2-(3,4-dichlorophenyl)-5-oxo-4-phenylmorpholin-2-yl]ethyl]-4-(3-fluorophenyl)piperidin-4-yl]acetamide and its pharmaceutically acceptable salts and of (+)-1′-[2-[4-benzoyl-2-(3,4-dichlorophenyl)-morpholin-2-yl]ethyl]-N,N-dimethyl-1,4′-bipiperidine-4′-carboxamide and its pharmaceutically acceptable salts, for the preparation of medicaments of use in the prevention and treatment of sexual dysfunction due to a general medical condition, particularly to a depressive disorder or to a major depressive disorder.

Also in particular, a subject of the present invention is the use of saredutant and of its pharmaceutically acceptable salts, of (+)-N-[1-[2-[2-(3,4-dichlorophenyl)-5-oxo-4-phenylmorpholin-2-yl]ethyl]-4-(3-fluorophenyl)piperidin-4-yl]acetamide and its pharmaceutically acceptable salts and of (+)-1′-[2-[4-benzoyl-2-(3,4-dichlorophenyl)-morpholin-2-yl]ethyl]-N,N-dimethyl-1,4′-bipiperidine-4′-carboxamide and its pharmaceutically acceptable salts, for the preparation of medicaments of use in the prevention and treatment of substance-induced sexual dysfunction.

Also in particular, a subject of the present invention is the use of saredutant and of its pharmaceutically acceptable salts, of (+)-N-[1-[2-[2-(3,4-dichlorophenyl)-5-oxo-4-phenylmorpholin-2-yl]ethyl]-4-(3-fluorophenyl)piperidin-4-yl]acetamide and its pharmaceutically acceptable salts and of (+)-1′-[2-[4-benzoyl-2-(3,4-dichlorophenyl)-morpholin-2-yl]ethyl]-N,N-dimethyl-1,4′-bipiperidine-4′-carboxamide and its pharmaceutically acceptable salts, for the preparation of medicaments of use in the prevention and treatment of unspecified sexual dysfunctions.

Saredutant and its pharmaceutically acceptable salts can be prepared according to the process described in patent EP 0 474 561 or that described in patent EP 0 698 601.
(+)-N-[1-[2-[2-(3,4-Dichlorophenyl)-5-oxo-4-phenylmorpholin-2-yl]ethyl]-4-(3-fluorophenyl)piperidin-4-yl]acetamide and its pharmaceutically acceptable salts can be prepared according to the process described in international application WO 2006/021 654.
(+)-1′-[2-[4-Benzoyl-2-(3,4-dichlorophenyl)morpholin-2-yl]ethyl]-N,N-dimethyl-1,4′-bipiperidine-4′-carboxamide and its pharmaceutically acceptable salts can be prepared according to the process described in international application WO 02/094 821.

According to another of its aspects, a subject of the present invention is a method of treatment or of prevention of sexual dysfunctions by administration of an appropriate dose of saredutant or of one of its pharmaceutically acceptable salts, or of (+)-N-[1-[2-[2-(3,4-dichlorophenyl)-5-oxo-4-phenylmorpholin-2-yl]ethyl]-4-(3-fluorophenyl)piperidin-4-yl]acetamide or of one of its pharmaceutically acceptable salts, or of (+)-1′-[2-[4-benzoyl-2-(3,4-dichlorophenyl)morpholin-2-yl]ethyl]-N,N-dimethyl-1,4′-bipiperidine-4′-carboxamide or of one of its pharmaceutically acceptable salts.

According to another of its aspects, a subject of the present invention is also the combination of a compound that is a neurokinin A NK2 antagonist according to the invention with another active ingredient of use in the treatment of sexual dysfunctions, such as, for example: sildenafil, vardenafil, tardalafil, alprostadil, apomorphine, midrodrine, moxisylite, phentolamine, aviptadil, testosterone, dapoxetine, tobolone; thus, the invention also relates to the pharmaceutical compositions containing this composition.

According to another aspect of the invention, the compound that is a neurokinin A NK2 receptor antagonist according to the invention and the other active ingredient according to the invention can be administered simultaneously, separately or sequentially.

The term “simultaneous use” is intended to mean the administration of the compounds of the composition according to the invention included in one and the same pharmaceutical form.

The term “separate use” is intended to mean the administration, at the same time, of the two compounds of the composition according to the invention, each included in a distinct pharmaceutical form.

The term “sequential use” is intended to mean the successive administration of the first compound of the composition of the invention, included in one pharmaceutical form, and then of the second compound of the composition according to the invention, included in a distinct pharmaceutical form.

In the case of this “sequential use” the amount of time that elapses between the administration of the first compound of the composition according to the invention and the administration of the second compound of the same composition according to the invention does not generally exceed 24 hours.

The unit pharmaceutical forms containing either just one of the compounds constituting the composition according to the invention, or the combination of the 2 compounds which can be employed in the various types of use described above may, for example, be suitable for oral, nasal, parenteral or transdermal administration.

Thus, in the case of a “separate use” and of a “sequential use”, two distinct pharmaceutical forms may be intended for the same route of administration or for a different route of administration (oral and transdermal or oral and nasal or parenteral and transdermal, etc.).

The invention therefore also relates to a kit containing the compound that is a neurokinin A NK2 receptor antagonist according to the invention and the other active ingredient according to the invention, in which said compound that is a neurokinin A NK2 receptor antagonist according to the invention and the other active ingredient according to the invention are in separate compartments and in similar or different packagings, and are intended to be administered simultaneously, separately or sequentially.

For its use as a medicament, the neurokinin A NK2 receptor antagonist, or one of its pharmaceutically acceptable salts, should be formulated in a pharmaceutical composition. In the pharmaceutical compositions of the present invention for oral, sublingual, subcutaneous, intramuscular, intravenous, transdermal or local administration, the active ingredient may be administered in particular in unit form, as a mixture with conventional pharmaceutical carriers, to animals and to human beings. The suitable administration forms include forms for oral administration, such as tablets, gel capsules, pills, powders, granules and oral solutions or suspensions, sublingual and buccal administration forms, and local, intracavernous, transdermal, intramuscular and intravenous administration forms. In the pharmaceutical compositions of the present invention, the active ingredient(s) is (are) generally formulated in dosage units. The dosage unit contains 2.5 to 500 mg, advantageously from 30 to 250 mg, preferably from 30 to 150 mg per dosage unit, for daily administrations, one or more times a day. Although these dosages are examples of average situations, there may be specific cases where higher or lower dosages are appropriate, such dosages also being part of the invention. According to the usual practice, the dosage suitable for each patient is determined by the physician according to the method of administration, and the age, weight and response of said patient.

Preferably, the neurokinin A NK2 receptor antagonist is administered orally, in a single intake.

The effects of saredutant have been determined during eight-week, multicentre, double-blind clinical studies versus placebo, in male or female adult patients, exhibiting a major depressive disorder. The patients received, orally, a dose of 100 mg per day of saredutant for eight weeks, in the form of gel capsules (Example 2).

he effects of saredutant compared with the placebo were evaluated using the CSFQ questionnaire (changes in sexual functioning questionnaire) comprising 14 items according to Clayton A H et al., Psychopharmacol. Bull., 1997, 33, 731-745.

It is known that antidepressants have a deleterious effect on the sexual activity of patients (Montejo-Gonzales A L et al.: J. Sex Marital Ther., 1997, 23(3), 176-194). Now, surprisingly, saredutant demonstrated a significant improvement (p<0.05) in the total CSFQ score between the last visit (schedule for the 56th day) and the first visit (before the beginning of the treatment) in comparison with the placebo group (p<0.05).

The table below indicates, for each group treated, the mean variation relative to the base value before treatment of the total CSFQ score and its variability between parentheses obtained over the analysis of all the studies. An increase in the total CSFQ score corresponds to an improvement in sexual function.

TABLE I
Total CSFQ score

| | Placebo (n = 551) | Saredutant (n = 553) |
|---|---|---|
| All patients | 1.36 (0.64) | 2.23 (0.64)* |

*p < 0.05 versus placebo in ANCOVA (analysis of covariance, including the base value of the total CSFQ score, the sex factor, and the factor studied, treated as a random factor), n = number of patients.

These studies show, surprisingly, a significant increase in the CSFQ score of the sexual dysfunctions in the patients treated with saredutant versus the patients receiving the placebo.

More particularly, a beneficial effect was observed on the following dysfunctions: reduced sexual desire and sexual arousal disorders.

The following nonlimiting examples describe examples of pharmaceutical compositions, of use for the use according to the invention of a neurokinin A NK2 receptor antagonist. Saredutant is used in monosuccinate form; (+)-N-[1-[2-[2-(3,4-dichlorophenyl)-5-oxo-4-phenylmorpholin-2-yl]ethyl]-4-(3-fluorophenyl)piperidin-4-yl]acetamide in hydrochloride form, and (+)-[1-[2-[4-benzoyl-2-(3,4-dichlorophenyl)-morpholin-2-yl]ethyl]-N,N-dimethyl-1,4′-bipiperidine-4′-carboxamide in dihydrochloride form.

### EXAMPLE 1

### Gel Capsule Containing 30 mg of Saredutant

| Saredutant (expressed as base) | 30.0 | mg |
|---|---|---|
| Lactose monohydrate (200 mesh) | QS 400.0 | mg |
| Sodium croscarmellose | 8.0 | mg |
| Magnesium stearate | 4.0 | mg |
| Purified water* | QS | |
| For an opaque gel capsule of size 0, fill to | 400.0 | mg |

*Evaporated at drying after wet granulation.

### EXAMPLE 2

### Gel Capsule Containing 100 mg of Saredutant

| Saredutant (expressed as base) | 100.0 | mg |
|---|---|---|
| Lactose monohydrate (200 mesh) | QS 400.0 | mg |
| Sodium croscarmellose | 8.0 | mg |
| Magnesium stearate | 4.0 | mg |
| Purified water* | QS | |
| For an opaque gel capsule of size 0, fill to | 400.0 | mg |

*Evaporated at drying after wet granulation.

### EXAMPLE 3

### Gel Capsule Containing 50 mg of (+)-N-[1-[2-[2-(3,4-dichlorophenyl)-5-oxo-4-phenylmorpholin-2-yl]ethyl]-4-(3-fluorophenyl)piperidin-4-yl]acetamide

| (Expressed as base) | 50.0 | mg |
|---|---|---|
| Lactose monohydrate (200 mesh) | 44.0 | mg |
| Hydroxypropylmethylcellulose | 2.0 | mg |
| Sodium carboxymethyl starch | 3.0 | mg |
| Magnesium stearate | 1.0 | mg |
| Purified water* | QS | |
| For an opaque gel capsule fill to | 100.0 | mg |

*Evaporated at drying after wet granulation.

### EXAMPLE 4

### Tablet Containing 50 mg of (+)-1′-[2-[4-benzoyl-2-(3,4-dichlorophenyl)morpholin-2-yl]ethyl]-N,N-dimethyl-1,4′-bipiperidine-4′-carboxamide

| | | |
|---|---|---|
| (expressed as base) | 50.0 | mg |
| Mannitol | 223.75 | mg |
| Sodium croscarmellose | 6.0 | mg |
| Maize starch | 15.0 | mg |
| Hydroxypropylmethylcellulose | 2.25 | mg |
| Magnesium stearate | 3.0 | mg |
| For a tablet containing at the end | 300.0 | mg |

What is claimed is:

1. A method for treating a sexual dysfunction in a patient, said method comprising administering to said patient a neurokinin A NK₂ receptor antagonist selected from:
saredutant;
(+)-N-[1-[2-[2-(3,4-dichlorophenyl)-5-oxo-4-phenylmorpholin-2-yl]ethyl]-4-(3-fluorophenyl)piperidin-4-yl]acetamide; and
(+)-1′-[2-[4-benzoyl-2-(3,4-dichlorophenyl)morpholin-2-yl]ethyl]-N,N-dimethyl-1,4′-bipiperidine-4′-carboxamide;
or a pharmaceutically acceptable salt thereof.

2. The method according to claim 1, wherein said neurokinin A NK₂ receptor antagonist is saredutant, or a pharmaceutically acceptable salt thereof.

3. The method according to claim 1, wherein said neurokinin A NK₂ receptor antagonist is (+)-N-[1-[2-[2-(3,4-dichlorophenyl)-5-oxo-4-phenylmorpholin-2-yl]ethyl]-4-(3-fluorophenyl)piperidin-4-yl]acetamide, or a pharmaceutically acceptable salt thereof.

4. The method according to claim 1, wherein said neurokinin A NK₂ receptor antagonist is (+)-1′-[2-[4-benzoyl-2-(3,4-dichlorophenyl)morpholin-2-yl]ethyl]-N,N-dimethyl-1,4′-bipiperidine-4′-carboxamide, or a pharmaceutically acceptable salt thereof.

5. The method according to claim 2, wherein said sexual dysfunction is sexual desire disorder.

6. The method according to claim 5, wherein said sexual desire disorder is reduced sexual desire.

7. The method according to claim 5, wherein said sexual desire disorder is sexual aversion.

8. The method according to claim 3, wherein said sexual dysfunction is sexual desire disorder.

9. The method according to claim 8, wherein said sexual desire disorder is reduced sexual desire.

10. The method according to claim 8, wherein said sexual desire disorder is sexual aversion.

11. The method according to claim 4, wherein said sexual dysfunction is sexual desire disorder.

12. The method according to claim 11, wherein said sexual desire disorder is reduced sexual desire.

13. The method according to claim 11, wherein said sexual desire disorder is sexual aversion.

14. The method according to claim 2, wherein said sexual dysfunction is a sexual arousal disorder.

15. The method according to claim 14, wherein said sexual arousal disorder is female sexual arousal disorder.

16. The method according to claim 14, wherein said sexual arousal disorder is male erectile disorder.

17. The method according to claim 3, wherein said sexual dysfunction is sexual arousal disorder.

18. The method according to claim 17, wherein said sexual arousal disorder is female sexual arousal disorder.

19. The method according to claim 17, wherein said sexual arousal disorder is male erectile disorder.

20. The method according to claim 4, wherein said sexual dysfunction is sexual arousal disorder.

21. The method according to claim 20, wherein said sexual arousal disorder is female sexual arousal disorder.

22. The method according to claim 20, wherein said sexual arousal disorder is male erectile disorder.

23. The method according to claim 2, wherein said sexual dysfunction is an orgasmic disorder.

24. The method according to claim 23, wherein said orgasmic disorder is female orgasmic disorder.

25. The method according to claim 23, wherein said orgasmic disorder is male orgasmic disorder.

26. The method according to claim 23, wherein said orgasmic disorder is premature ejaculation.

27. The method according to claim 3, wherein said sexual dysfunction is an orgasmic disorder.

28. The method according to claim 27, wherein said orgasmic disorder is female orgasmic disorder.

29. The method according to claim 27, wherein said orgasmic disorder is male orgasmic disorder.

30. The method according to claim 27, wherein said orgasmic disorder is premature ejaculation.

31. The method according to claim 4, wherein said sexual dysfunction is an orgasmic disorder.

32. The method according to claim 31, wherein said orgasmic disorder is female orgasmic disorder.

33. The method according to claim 31, wherein said orgasmic disorder is male orgasmic disorder.

34. The method according to claim 31, wherein said orgasmic disorder is premature ejaculation.

35. The method according to claim 2, wherein said sexual dysfunction is a painful sexual disorder.

36. The method according to claim 35, wherein said painful sexual disorder is dyspareunia.

37. The method according to claim 35, wherein said painful sexual disorder is vaginismus.

38. The method according to claim 3, wherein said sexual dysfunction is a painful sexual disorder.

39. The method according to claim 38, wherein said painful sexual disorder is dyspareunia.

40. The method according to claim 38, wherein said painful sexual disorder is vaginismus.

41. The method according to claim 4, wherein said sexual dysfunction is a painful sexual disorder.

42. The method according to claim 41, wherein said painful sexual disorder is dyspareunia.

43. The method according to claim 41, wherein said painful sexual disorder is vaginismus.

44. The method according to claim 2, wherein said sexual dysfunction is due to a general medical condition.

45. The method according to claim 44, wherein said general medical condition is a depressive disorder.

46. The method according to claim 44, wherein said general medical condition is a major depressive disorder.

47. The method according to claim 3, wherein said sexual dysfunction is due to a general medical condition.

48. The method according to claim 47, wherein said general medical condition is a depressive disorder.

49. The method according to claim 47, wherein said general medical condition is a major depressive disorder.

50. The method according to claim 4, wherein said sexual dysfunction is due to a general medical condition.

51. The method according to claim 50, wherein said general medical condition is a depressive disorder.

52. The method according to claim 50, wherein said general medical condition is a major depressive disorder.

53. The method according to claim 2, wherein said sexual dysfunction is a substance-induced sexual dysfunction.

54. The method according to claim 3, wherein said sexual dysfunction is a substance-induced sexual dysfunction.

55. The method according to claim 4, wherein said sexual dysfunction is a substance-induced sexual dysfunction.

56. The method according to claim 2, wherein said sexual dysfunction is an unspecified sexual dysfunction.

57. The method according to claim 3, wherein said sexual dysfunction is an unspecified sexual dysfunction.

58. The method according to claim 4, wherein said sexual dysfunction is an unspecified sexual dysfunction.

59. A pharmaceutical composition comprising a first compound and a second compound, wherein:
said first compound is a neurokinin A NK₂ receptor antagonist selected from saredutant, (+)-N-[1-[2-[2-(3,4-dichlorophenyl)-5-oxo-4-phenylmorpholin-2-yl]ethyl]-4-(3-fluorophenyl) [symbol] piperidin-4-yl]acetamide, and (+)-1′-[2-[4-benzoyl-2-(3,4-dichlorophenyl)morpholin-2-yl]ethyl]-N,N-dimethyl-1,4′-bipiperidine-4′-carboxamide; or a pharmaceutically acceptable salt thereof; and
said second compound is selected from sildenafil, vardenafil, tardalafil, alprostadil, apomorphine, midrodrine, moxisylite, phentolamine, aviptadil, testosterone, dapoxetine and tobolone.

60. A method for treating a sexual dysfunction in a patient, said method comprising administering to said patient a pharmaceutical composition according to claim 59.

61. A method for treating a sexual dysfunction in a patient, said method comprising administering to said patient a first compound and a second compound, wherein:
aid first compound is a neurokinin A NK₂ receptor antagonist selected from saredutant, (+)-N-[1-[2-[2-(3,4-dichlorophenyl)-5-oxo-4-phenylmorpholin-2-yl]ethyl]-4-(3-fluorophenyl) [symbol] piperidin-4-yl]acetamide, and (+)-1′-[2-[4-benzoyl-2-(3,4-dichlorophenyl)morpholin-2-yl]ethyl]-N,N-dimethyl-1,4′-bipiperidine-4′-carboxamide; or a pharmaceutically acceptable salt thereof; and
said second compound is selected from sildenafil, vardenafil, tardalafil, alprostadil, apomorphine, midrodrine, moxisylite, phentolamine, aviptadil, testosterone, dapoxetine and tobolone.

62. The method according to claim 61, wherein said first compound and said second compound are administered simultaneously, separately, or sequentially.

63. A kit containing a first compound and a second compound, wherein:
said first compound is a neurokinin A NK₂ receptor antagonist selected from saredutant, (+)-N-[1-[2-[2-(3,4-dichlorophenyl)-5-oxo-4-phenylmorpholin-2-yl]ethyl]-4-(3-fluorophenyl) [symbol] piperidin-4-yl]acetamide, and (+)-1′-[2-[4-benzoyl-2-(3,4-dichlorophenyl)morpholin-2-yl]ethyl]-N,N-dimethyl-1,4′-bipiperidine-4′-carboxamide; or a pharmaceutically acceptable salt thereof; and
said second compound is selected from sildenafil, vardenafil, tardalafil, alprostadil, apomorphine, midrodrine, moxisylite, phentolamine, aviptadil, testosterone, dapoxetine and tobolone;
said first compound and said second compound are in separate compartments and in similar or different packagings; and
said first compound and said second compound are intended to be administered simultaneously, separately or sequentially.
